(19) United States
(12) Patent Application Publication
(10) Pub. No.: US 2022/0274931 A1
(43) Pub. Date: Sep. 1, 2022

(54) PROTECTED TRIAZABUTADIENE COMPOSITIONS FOR CELLULAR STUDIES IN INTACT BIOLOGICAL SYSTEMS

(71) Applicant: ARIZONA BOARD OF REGENTS ON BEHALF OF THE UNIVERSITY OF ARIZONA, Tucson, AZ (US)

(72) Inventors: John C. Jewett, Tucson, AZ (US); Natasha Cornejo, Tucson, AZ (US); Bismark Amofah, Tucson, AZ (US); Lindsay E. Guzman, Tucson, AZ (US); W Mudiyanselage Anjalee Wijetunge, Tucson, AZ (US); Abigail Shepard, Tucson, AZ (US); Danielle Johnson, Tucson, AZ (US)

(21) Appl. No.: 17/751,474

(22) Filed: May 23, 2022

Related U.S. Application Data
(63) Continuation-in-part of application No. 16/321,605, filed on Jan. 29, 2019, now Pat. No. 11,339,129.
Application No. 16/321,605 is a continuation of application No. 15/427,988, filed on Feb. 8, 2017, now Pat. No. 10,125,105.
Application No. 15/427,988 is a continuation-in-part of application No. 15/317,894, filed on Dec. 9, 2016, now Pat. No. 10,047,061.
Application No. 15/317,894 is a continuation-in-part of application No. 15/224,446, filed on Jul. 29, 2016, now Pat. No. 9,593,080.

(51) Int. Cl. C07D 233/88; C09K 11/06; C07D 403/12; C07D 405/12; C07D 413/14; C07H 21/00; C09B 26/06; C09B 55/00; C09J 163/00; G01N 33/569; G01N 33/68; A61K 47/54; A61K 49/00

(52) U.S. Cl. CPC C07D 233/88; C09K 11/06; C07D 403/12; C07D 405/12; C07D 413/14; C07H 21/00; C09B 26/06; C09B 55/003; C09J 163/00; G01N 33/56983; G01N 33/6845; A61K 47/545; A61K 49/0021; C09K 2211/1048

(57) ABSTRACT

Protected triazabutadiene molecules, such as those according to Formula D or variations thereof, as probes for cellular studies in intact biological systems. The protected triazabutadiene probes selectively release benzene diazonium ions (BDIs) intracellularly, providing a tool for accessing and/or labeling intracellular proteins or molecules prior to cell lysis. The present invention also includes methods for synthesizing the protected triazabutadienes, the protected triazabutadienes themselves, and methods of use.

### CROSS-REFERENCES TO RELATED APPLICATIONS

This application is a continuation-in-part and claims benefit of U.S. patent application Ser. No. 16/321,605 filed Jan. 29, 2019, which is a 371 of PCT/US17/44737 filed Jul. 31, 2017, which is a continuation and claims benefit of U.S. patent application Ser. No. 15/427,988 filed Feb. 8, 2017, now U.S Pat. No. 10,125,105, a continuation-in-part and claims benefit of U.S. patent application Ser. No. 15/317,894 filed Dec. 9, 2016, now U.S. Pat. No. 10,047,061, and a continuation-in-part and claims benefit of U.S. patent application Ser. No. 15,224,446 filed Jul. 29, 2016, now U.S. Pat. No. 9,593,080, the specifications of which are incorporated herein in their entirety by reference.

### STATEMENT REGARDING FEDERALLY SPONSORED RESEARCH OR DEVELOPMENT

This invention was made with government support under Grant No. 1552568, awarded by National Science Foundation. The government has certain rights in the invention.

### BACKGROUND OF THE INVENTION

### Field of the Invention

The present invention relates to probes for intracellular use, more particularly to probes that undergo reduction-mediated deprotection to deliver a benzene diazonium ion into cells, e.g., for the purpose of cellular studies in intact biological systems.

### Background Art

Selectivity in biological systems comes from a complex interplay of location, interactions, and reactivity. Covalent small-molecule probes offer great potential in the development of chemical tools to study intracellular proteins. A challenge within this area is in garnering selectivity associated with location. For example, in order to gain accessibility to intracellular proteins, small-molecule labeling strategies have relied on working with cell lysate, yet key contextual interactions associated with localization within the cell are lost during lysis.

Aryl diazonium ions are known for their selective reactivity with the electron-rich aromatic tyrosine side chain. But aryl diazonium ions for use as probes have suffered from a lack of deliverability since they have short half-lives and are generally unstable.

The present invention provides probes, e.g., protected triazabutadiene probes, that selectively release benzene diazonium ions (BDIs) intracellularly, providing a tool for cellular studies in intact biological systems, e.g., a means for accessing and/or labeling intracellular proteins or molecules prior to cell lysis. The present invention is not limited to reactivity with tyrosine and may include any other appropriate moiety or molecule such as but not limited to histidine.

### SUMMARY OF THE INVENTION

The present invention features compositions for selective intracellular delivery of a diazonium species. The diazonium species labels a tyrosine or histidine residue of a protein in cellulo. For example, the compositions are configured to selectively release a diazonium species upon exposure to a high pH. The compositions herein can be taken up intracellularly.

In some embodiments, the high pH is a pH of 9 or higher. In some embodiments, the high pH is a pH of 9.2 or higher. In some embodiments, the high pH is a pH of 9.4 or higher. In some embodiments, the high pH is a pH of 9.5 or higher. In some embodiments, the high pH is a pH of 9.6 or higher. In some embodiments, the high pH is a pH of 9.8 or higher. In some embodiments, the high pH is a pH of 10 or higher. In some embodiments, the high pH is a pH of 10.2 or higher. In some embodiments, the high pH is a pH of 10.5 or higher. In some embodiments, the high pH is a pH of 11 or higher. In some embodiments, the high pH is a pH of 11.5 or higher. In some embodiments, the high pH is a pH of 12 or higher. The present invention is not limited to the aforementioned pH values. For example, depending on the side groups of the compositions and the application, in some embodiments, the high pH is 8.9, 8.8, 8.7, 8.6, 8.5, 8.4, 8.3, 8.2, 8.1, 8.0, etc.

The present invention also features compositions according to Formula D. Formula D is shown below.

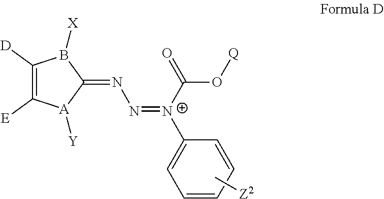

In some embodiments, Z² is a NHS ester, a Cu click reagent, a Cu free click reagent, a bioorthogonal handle, or a drug. In some embodiments, Q is —(CH₂)₂S—, an enzymatically cleavable moiety, a self-immolative linker, a quinone methide forming cascade reaction, or a Grob-fragmentation related cleavable linker.

Without wishing to limit the present invention to any theory or mechanism, it is believed that Q helps enable release in the presence of a particular environment or in the presence of a particular enzyme. It can also help enable cell uptake, cell targeting, cell localization, etc. Without wishing to limit the present invention to any theory or mechanism it is believed that Q can help make the compositions more water soluble, e.g., because of the charge, which helps draw the compounds into the cell.

In some embodiments, A is N, S, or O; and B is N, S, or O. In some embodiments, if A is N, B could be N, S or O. In some embodiments, if B is N, A could be N, S, or O. In some embodiments, if A is S or O, B is N. In some embodiments, if B is S or O, A is N. The present invention is not limited to the aforementioned structures.

In some embodiments, D is H, —CH=CH—CH=E— (e.g., see Formula III and IV in FIG. 1A), halides, cyano, sulfonates, alkyl chain, or trifluoromethyl. In some embodiments, E is H, —CH=CH—CH=D— (see Formula III and IV in FIG. 1A), halides, cyano, sulfonates, alkyl chain, or trifluoromethyl.

In some embodiments, X is —R1-K1, wherein —R1=alkanes and K=sulfonate, phosphate, or a quaternary ammonium cation, or an alkyl, aryl or propargylic containing moiety that can facilitate coupling to other azides via [3+2]cycloaddition chemistry. IN some embodiments, X is non-existent if A is S. In some embodiments, Y is a tri-substituted aryl group or an alkyl substituent. In some embodiments, Y is non-existent if B is S. In some embodiments, the tri-substituted aryl group of Y comprises mesityl, a

NHS-ester moiety; an oligonucleotide; a peptide; a fluorescence quencher; a pro-fluorophore; an alkyne; a triazene; an aldehyde; an amine; an aminooxy; a halogen; or a combination thereof.

In some embodiments, one, or a combination of, or all of X, Y, and Q comprise a biological directing group. In some embodiments, the biological directing group is a triphenylphosphonium for directing the composition to the mitochondria or a folate for inducing cellular uptake via the folate receptor.

Referring to the structures above, NHS esters, Cu click reagents, Cu free click reagents, appropriate drugs, bioorthogonal handles, enzymatically cleavable moieties, self-immolative linkers, quinone methods, Grob-fragmentation related cleavable linkers, alkanes, sulfonates, phosphates, quaternary ammonium cations, alkyl groups, aryl groups, halides, cyano, sulfonates, alkyl chain, or trifluoromethyl groups, propargylic moieties, tri-substituted aryl groups, etc. are well known to one of ordinary skill in the art and can be readily identified in the literature.

The present invention also features compositions according to Formula E. Formula E is shown below.

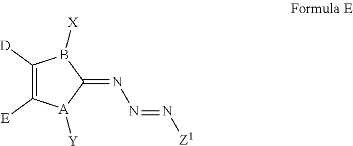

In some embodiments, A is N, S, or O; and B is N, S, or O. In some embodiments, if A is N, B could be N, S or O. In some embodiments, if B is N, A could be N, S, or O. In some embodiments, if A is S or O, B is N. In some embodiments, if B is S or O, A is N. The present invention is not limited to the aforementioned structures.

In some embodiments, D is H, —CH=CH—CH=— (e.g., see Formula III and IV in FIG. 1A), halides, cyano, sulfonates, alkyl chain, or trifluoromethyl. In some embodiments, E is H, —CH=CH—CH=D— (see Formula III and IV in FIG. 1A), halides, cyano, sulfonates, alkyl chain, or trifluoromethyl. In some embodiments, D and/or E are other side groups, e.g., side groups disclosed herein or other appropriate side groups.

In some embodiments, X is —R1-K1, wherein —R1=alkanes and K=sulfonate, phosphate, or a quaternary ammonium cation, or an alkyl, aryl or propargylic containing moiety that can facilitate coupling to other azides via [3+2]cycloaddition chemistry. IN some embodiments, X is non-existent if B is S. In some embodiments, Y is a tri-substituted aryl group or an alkyl substituent. In some embodiments, Y is non-existent if A is S. In some embodiments, the tri-substituted aryl group of Y comprises mesityl, a NHS-ester moiety; an oligonucleotide; a peptide; a fluorescence quencher; a pro-fluorophore; an alkyne; a triazene; an aldehyde; an amine; an aminooxy; a halogen; or a combination thereof.

In some embodiments, Z¹ is a polymerization residue, a phenyl group, a substituted phenyl group, or —COO—Q. In some embodiments, Z¹ is a pair of compounds as is shown in Formula D wherein two compounds are bound to the N1 nitrogen. In some embodiments, Z is a pair of compounds, wherein the first compound is a phenyl group or substituted phenyl group (e.g., phenyl-Z²) and the second compound is —COO—Q. In some embodiments, Z² is a NHS ester, a Cu click reagent, a Cu free click reagent, a bioorthogonal handle, or a drug. In some embodiments, Q is —(CH₂)₂S—, an enzymatically cleavable moiety, a self-immolative linker, a quinone methide forming cascade reaction, or a Grob-fragmentation related cleavable linker.

In some embodiments, Z¹ is configured to add charge to N1 nitrogen. The charge helps enable the composition to be taken up by a cell.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1A shows non-limiting examples of triazabutadiene molecules.

FIG. 1B shows examples of additional structures of triazabutadienes (see Formula B and Formula C compared to Formula A).

FIG. 2 shows triazabutadiene molecules undergoing decomposition to diazonium salts (and cyclic guanidine species). Note the reaction/equilibrium arrows are not to scale.

FIG. 3 shows triazabutadiene releasing a diazonium ion (benzene diazonium ion, or BDI) under mild conditions at physiological pH. The N1 position of the triazabutadienee can be protected via a covalent modification to prevent BDI release. The protected triazabutadiene can then be deprotected to rescue triazabutadiene reactivity and BDI release. Azo-tyrosine adducts are unable to be phosphorylated. Conversely, phosphorylated tyrosine residues cannot react with BDIs. BDI conjugation to tyrosine is inversely related to tyrosine phosphorylation.

FIG. 4A shows a suggested mechanism of compound (2) in cells, wherein compound 2 will release the BDI in <1 min, which can then either react with proteins extracellularly, diffuse through the cell membrane and react with intracellular proteins, or undergo hydrolysis.

FIG. 4B shows the concentration dependence of compound 2 with HEK293T cells, wherein an increase in global p-Tyr is seen at a concentration of 500 μM and above.

FIG. 4C shows a time dependent experiment with 500 μM 2, wherein an increase in global p-Tyr is observed at 120 min. While BDI conjugation to histidine is known, the figure only shows tyrosine conjugation for clarity.

FIG. 5 shows an example of synthesis of a protected triazabutadiene.

FIG. 6A shows a suggested mechanism for compound 3 in cells. Intracellular delivery of 3 triggers a reduction of the disulfide mimic and subsequent deprotection, delivering 2 intracellularly, which will then release a BDI.

FIG. 6B shows a suggested mechanism for control compounds 6 and 7, which should both get transferred intracellularly, but should remain in their protected form, never releasing a BDI.

FIG. 6C shows the concentration dependence of 3 with HEK293T cells, wherein an increase in global p-Tyr is observed at 250 and 500 μM.

FIG. 6D shows a time-dependent experiment for 3 at 500 μM with HEK293 cells, wherein an increase in global p-Tyr is observed at 30 min

FIG. 6E shows control compounds 6 and 7 tested alongside 2 and 3, wherein control compounds have no effect on global p-Tyr, and compound 3 increases global p-Tyr at 30 min, consistent with previous results.

FIG. 7 shows the structure of Formula D, e.g., a non-limiting example of a protected triazabutadiene molecule. Non-limiting examples of Q are shown in FIG. 9A, FIG. 9B, and FIG. 9C.

FIG. 8 shows non-limiting examples of protected triazabutaidene molecules that enable further conjugation to other molecules, e.g., biomolecules.

FIGS. 9A-9C show non-limiting examples of Q and a range of mechanisms by which triazabutadiene protecting groups can be removed.

FIG. 10 shows the structure of Formula E.

### DETAILED DESCRIPTION OF THE INVENTION

I. Triazabutadiene Molecules

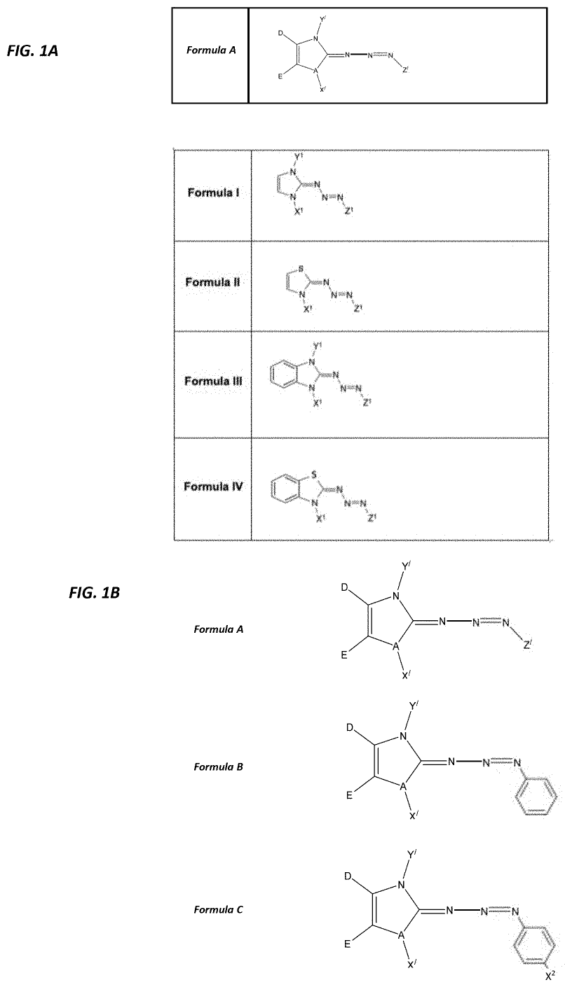

The present invention features triazabutadiene molecules. Non-limiting examples of formulas for triazabutadiene molecules of the present invention are shown in FIG. 1A. For example, in some embodiments, triazabutadienes are according to Formula A. Examples of Formula A are shown as Formula I, II, III, and IV. The present invention is not limited to Formula A, Formula I, Formula II, Formula III, and Formula IV. Referring to FIG. 1A, in some embodiments, A=S, O, or N. In some embodiments, D=H, —CH=CH—CH=E—, halides, cyano, sulfonates, alkyl chain, or trifluoromethyl. In some embodiments, E=H, —CH=CH—CH=D—, halides, cyano, sulfonates, alkyl chain, or trifluoromethyl.

In some embodiments, X¹ is a moiety conferring water solubility. In some embodiments, Y¹ is a tri-substituted aryl group. In some embodiments, the Y¹ (e.g., the tri-substituted aryl group) comprises a NHS-ester moiety (e.g., for protein linkage); an oligonucleotide; a peptide; a fluorescence quencher; a pro-fluorophore; an alkyne (e.g., for click chemistry); a triazene (e.g., from click reaction); the like, or a combination thereof. In some embodiments, Y¹ comprises an aldehyde; an amine (e.g., Fmoc protected), aminooxy, halogen (e.g., radio isotope); the like, or a combination thereof. In some embodiments, Z¹ is an optionally substituted aryl. In some embodiments, Z¹ comprises a NHS-ester moiety; an oligonucleotide; a peptide; a fluorescence quencher; a pro-fluorophore; a biologically active acid labile compound; a prodrug comprising a phenolic functional group; releasable cargo; an alkyne (e.g., for click chemistry); a triazene (e.g., from click reaction); a polymerization residue (e.g., epoxide, polystyrene, alpha-beta-unsaturated ester acrylate, polyacrylamide, an amine, etc.), the like, or a combination thereof. In some embodiments, Z¹ comprises an aldehyde; an amine (e.g., Fmoc protected), aminooxy, halogen (e.g., radio isotope); the like, or a combination thereof.

In some embodiments, X¹ may comprise a functional group that confers water solubility. In some embodiments, X¹ comprise a moiety of the formula —R¹—Q¹, wherein R¹ is C₁₋₆ alkylene, and Q¹ is sulfate, sulfonate, phosphate, a quaternary ammonium cation, or an alkyl, aryl or propargylic containing moiety that can facilitate coupling to other azides via [3+2] cycloaddition chemistry. In some embodiments, X¹ is a moiety of the formula —R¹—Q¹, wherein R¹ is an alkane, e.g., C₁₋₆ alkylene. In some embodiments, Q¹ is sulfate (e.g., —(O)ₙPO₃R^{a}, where n is 0 or 1, and R^{a} is C1-6 alkyl or typically H), phosphate (e.g., —(O)ₙPO₃R^{a}, where n is 0 or 1, and R^{a} is C1-6 alkyl or typically H), or a quaternary ammonium cation (e.g., —[NR^{a}R^{b}Rc]⁺, where each of R^{a}, R^{b}, and R^{c} is independently H or C₁₋₆ alkyl). As used herein, the term “alkyl” refers to a saturated linear monovalent hydrocarbon moiety of one to twelve, typically one to six, carbon atoms or a saturated branched monovalent hydrocarbon moiety of three to twelve, typically three to six, carbon atoms. Examples of alkyl groups include, but are not limited to, methyl, ethyl, n-propyl, 2-propyl, tert- butyl, pentyl, and the like. The term “alkylene” refers to a saturated linear divalent hydrocarbon moiety of one to twelve, typically one to six, carbon atoms or a branched saturated divalent hydrocarbon moiety of three to twelve, typically three to six, carbon atoms. Examples of alkylene groups include, but are not limited to, methylene, ethylene, propylene, butylene, pentylene, and the like.

Triazabutadiene molecules of the present invention are readily soluble in water. In some embodiments, the solubility of the triazabutadiene molecules in water is at least 23 g/L of water (50 mM). In some embodiments, the triazabutadiene molecules are stable in pH 7.4 phosphate buffer. The phosphate buffer solutions are commercially available or can be prepared, for example, as described in http://cshprotocols.cshlp.org/content/2006/1/pdb.rec8247. In some instances, the half-life of the triazabutadiene molecules of the present invention in pH 7.4 phosphate buffer solution is at least 24 hours.

Stability of the triazabutadiene molecule can be measured in various ways. In some embodiments, stability is measured by the half-life of the molecule (or the half-life of the molecule in a particular buffer at a particular pH). In some embodiments, the molecule has a half-life of at least 12 hours in a pH 7.4 buffer. In some embodiments, the molecule has a half-life of at least 24 hours in a pH 7.4 buffer. In some embodiments, the molecule has a half-life of at least 36 hours in a pH 7.4 buffer. In some embodiments, the triazabutadiene molecule has a half-life of at least 8 hours. In some embodiments, the triazabutadiene molecule has a half-life of at least 10 hours. In some embodiments, the triazabutadiene molecule has a half-life of at least 12 hours. In some embodiments, the triazabutadiene molecule has a half-life of at least 20 hours. In some embodiments, the triazabutadiene molecule has a half-life of at least 24 hours. In some embodiments, the triazabutadiene molecule has a half-life of at least 30 hours. In some embodiments, the triazabutadiene molecule has a half-life of at least 36 hours. The present invention is not limited to the aforementioned examples of stability measurements.

Without wishing to limit the present invention to any theory or mechanism, it is believed that the triazabutadiene molecules of the present invention are advantageous because the triazabutadiene molecules can be easily modified (e.g., various different functional groups can be easily used as X¹, Y¹, or Z¹ (see FIG. 1A). And, the release of the diazonium species following triazabutadiene molecule breakdown (via certain mechanisms, as described below) provides a functional group that can be taken advantage of in various applications. Also, it may be considered advantageous that the breakdown of the triazabutadiene molecule is irreversible.

FIG. 1B shows non-limiting examples of structures of triazabutadienes, e.g., those adapted for click chemistry, e.g., Formula B and Formula C. In some embodiments, X¹ comprises an alkyne handle. In some embodiments, X² comprises an alkyne handle. In some embodiments, X¹ comprises an azide handle. In some embodiments, X² comprises an azide handle. In some embodiments, the clickable triazabutadiene is according to Formula B, wherein X¹ comprises a terminal alkyne handle. In some embodiments, the triazabutadiene is according to Formula C wherein X¹ comprises a terminal alkyne. In some embodiments, the triazabutadiene is according to Formula C wherein X² comprises a terminal alkyne handle. In some embodiments, the triazabutadiene is according to Formula C wherein both X¹ and X² comprise a terminal alkyne handle. In some embodiments, A=S, O, or N; D=H, —CH=CH—CH=E—, halides, cyano, sulfonates, alkyl chain, or trifluoromethyl. In some embodiments, E=H, —CH=CH—CH=D—, halides, cyano, sulfonates, alkyl chain, or trifluoromethyl. Note in some embodiments, the X¹ and Y¹ may be switched. For example, in some embodiments, A is sulfur, and the alkyne is branched off of the other nitrogen.

II. Cleavage of Triazabutadiene Molecules

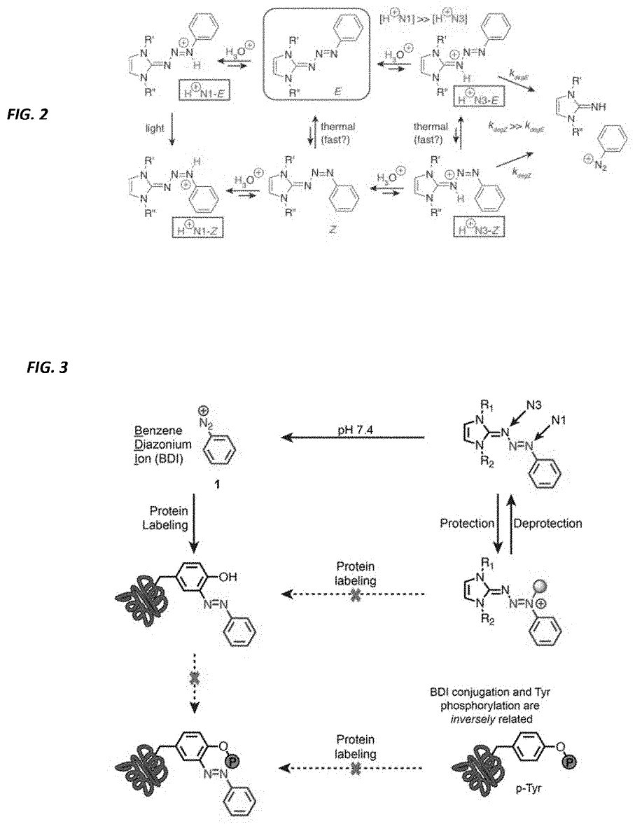

The present invention shows that triazabutadiene molecules may break down in the presence of water to generate reactive aryl diazonium compounds. For example, FIG. 2 shows that triazabutadiene molecules of the present invention can undergo decomposition to diazonium salts (reactive aryl diazonium compounds) and cyclic guanidine species. Aryl diazonium compounds can react with electron-rich aryl rings (e.g., aryl species wherein the bond of interest is a nitrogen-carbon bond; indoles, anilines, phenol-containing compounds such as resorcinol or tyrosine, etc.) to form stable azobenzene linkages (e.g., an aryl azo dye, e.g., Sudan Orange). (Note the present invention is not limited to the aforementioned phenol-containing species. In some embodiments, imidazole compounds (e.g., purine bases like guanine) may be used in lieu of a phenol-containing compound.) The diazonium species may not necessarily react with an electron-rich aryl rings compound (e.g., phenol species), for example if a phenol species is not present. The diazonium species may irreversibly extrude nitrogen gas to generate an aryl cation, which will rapidly be quenched by solvating water, thus synthesizing a new phenolic compound (e.g., HO—Ph, wherein Ph refers to the phenyl ring); thus, the diazonium portion of the triazabutadiene molecule may function as a masked hydroxyl group.

In some embodiments, the triazabutadiene molecules are acid labile, e.g., unstable at particular pH levels. For example, decreases in pH increase the rate at which the triazabutadiene molecules break down (the half life of the molecule decreases). In some embodiments, the triazabutadiene molecules are unstable at low (lowered) pH levels (e.g., lowered pH as compared to a particular pH that the molecule may be stored at, e.g., a pH wherein the molecule has a particular desired half life). Low pH levels, in some examples, may be a sub-physiological pH (7.4 or less). In some embodiments, the triazabutadiene molecules are (more) unstable at pH 7.0 or less, pH 6.8 or less, pH 6.5 or less, pH 6.2 or less, pH 6.0 or less, pH 5.8 or less, pH 5.6 or less, pH 5.5 or less, pH 5.2 or less, pH 5.0 or less, etc.

The term ‘low pH” may refer to several different pH levels. Since the functional groups attached to the molecule (e.g., see X¹, Y¹, Z¹ of Formula I) affect the stability of the molecule (as well as water solubility), the pH that is necessary to increase the rate of breakdown of the triazabutadiene molecule (e.g., the “lowered pH”) may be different for different molecules. In some embodiments, the low pH is a pH of 7.4 or less. In some embodiments, the low pH is a pH of 7.2 or less. In some embodiments, the low pH is a pH of 7.0 or less. In some embodiments, the low pH is a pH of 6.8 or less. In some embodiments, the low pH is a pH of 6.6 or less. In some embodiments, the low pH is a pH of 6.6 or less. In some embodiments, the low pH is a pH of 6.6 or less. In some embodiments, the low pH is a pH of 6.5 or less. In some embodiments, the low pH is a pH of 6.4 or less. In some embodiments, the low pH is a pH of 6.2 or less. In some embodiments, the low pH is a pH of 6.0 or less. In some embodiments, the low pH is a pH of 5.8 or less. In some embodiments, the low pH is a pH of 5.5 or less. In some embodiments, the low pH is a pH of 5.0 or less.

In some embodiments, the triazabutadiene molecules can break down without the presence of the low pH (the molecules have half lives); however, in some embodiments, a lowered pH enhances the reaction (e.g., increases the rate of reaction). As such, a low pH may or may not be used with the molecules and/or methods of the present invention. In some embodiments, the triazabutadiene molecule has a half-life of no more than 1 hour in a pH 7.4 aqueous solution. In some embodiments, the triazabutadiene molecule has a half-life of no more than 30 minutes in a pH 7.4 aqueous solution. In some embodiments, the triazabutadiene molecule has a half-life of no more than 15 minutes in a pH 7.4 aqueous solution.

The present invention also features methods of breaking down triazabutadiene molecules. In some embodiments, the method comprises subjecting the molecule to water. In some embodiments, the method comprises subjecting the molecule to a low pH (e.g., a low pH that is appropriate for the molecule, e.g., a lowered pH that increases the rate at which the triazabutadiene molecule breaks down).

In some embodiments, the reaction of the triazabutadiene molecule to the diazonium species occurs in water within 10 seconds minutes. In some embodiments, the reaction of the triazabutadiene molecule to the diazonium species occurs in water within 30 seconds minutes. In some embodiments, the reaction of the triazabutadiene molecule to the diazonium species occurs in water within 1 minute. In some embodiments, the reaction of the triazabutadiene molecule to the diazonium species occurs in water within 5 minutes. In some embodiments, the reaction of the triazabutadiene molecule to the diazonium species occurs in water within 10 minutes. In some embodiments, the reaction of the triazabutadiene molecule to the diazonium species occurs in water within 15 minutes. In some embodiments, the reaction of the triazabutadiene molecule to the diazonium species occurs in water within 20 minutes. In some embodiments, the reaction of the triazabutadiene molecule to the diazonium species occurs in water within 25 minutes. In some embodiments, the reaction of the triazabutadiene molecule to the diazonium species occurs in water within 30 minutes. In some embodiments, the reaction of the triazabutadiene molecule to the diazonium species occurs in water within 45 minutes. In some embodiments, the reaction of the triazabutadiene molecule to the diazonium species occurs in water within 60 minutes.

In some embodiments, the diazonium species may be visually differentiated from the triazabutadiene species, e.g., the diazonium species is visually distinct (e.g., a different color) from the triazabutadiene molecule. If applicable, in some embodiments, the aryl azo dye may be visually differentiated from the triazabutadiene species and the diazonium species, e.g., the aryl azo dye is visually distinct (e.g., a different color) from the triazabutadiene species and the diazonium species.

Given the possibility that the aryl azo dye is visually distinct from the triazabutadiene molecule (and/or the diazonium species), the present invention also features methods of producing a visually detectable molecule. In some embodiments, the method comprises providing a triazabutadiene molecule according to the present invention and subjecting the triazabutadiene molecule to water and/or a low pH (or light as discussed below, or light and low pH, etc.). The low pH (or light, or light and low pH, etc.) initiates (e.g., increases the rate of) the irreversible reaction to produce the diazonium species and the cyclic guanidine species. As previously discussed, the diazonium species may be visually distinct from the triazabutadiene molecule; therefore the reaction produces a visually detectable molecule.

Other mechanisms may be used to break down triazabutadiene molecules of the present invention. For example, in some embodiments, reducing conditions increase the rate at which the triazabutadiene molecules break down. Thus, the present invention also features methods of reductive cleavage of triazabutadiene molecules. For example, triazabutadiene molecules (e.g., triazabutadiene scaffolds) may be readily cleaved using reducing agents such as but not limited to sodium dithionite (sodium hydrosulfite) (Na₂S₂O₄). In some embodiments, the reducing agent comprises lithium aluminum hydride, sodium borohydride, or the like. In some embodiments, electrochemical reduction may be used in accordance with the present invention. Reductive cleavage of the triazabutadiene molecules provides a urea functionality and a terminal aryl triazene. In some embodiments, the aryl triazene is further reduced in the presence of an excess reducing agent (e.g., sodium dithionite). In some embodiments, the reduction can be observed visually by the change in color of a solution. For example, there may be a subtle change of yellows that results from a loss of a shoulder in UV/vis spectrum.

In some embodiments, the ratio of the concentration of the triazabutadiene to the reducing agent is about 1:1. In some embodiments, the ratio of the concentration of the triazabutadiene to the reducing agent is about 1:2. The present invention is not limited to the aforementioned ratios. For example, in some embodiments, the ratio of the concentration of the triazabutadiene to the reducing agent is about 2:3, 4:5, etc. The present invention is not limited to the aforementioned ratio of concentrations.

In some embodiments, the reduction can occur within about 10 minutes, within about 15 minutes, within about 20 minutes, within about 25 min, within about 30 min, etc., at room temperature. Without wishing to limit the present invention to any theory or mechanism, it is believed that reductive cleavage of the triazabutadiene molecules is advantageous because it can occur rapidly (e.g., within 10 minutes, within 15 minutes). Also, the triazabutadiene molecules that are highly stable in acid (e.g., a p-CN derived triazabutadiene) may still be susceptible to reducing conditions.

In some embodiments, reductive cleavage of triazabutadiene molecules may also be used to cleave unreacted triazabutadienes that did not undergo diazonium formation/reaction chemistry that is associated with a drop in pH (or other mechanism) as described above (a sort of quench for the pH chemistry).

In some embodiments, light increases the rate at which the triazabutadiene molecule breaks down (into the cyclic guanidine species and the diazonium species). The present invention features triazabutadienes that, upon photo-irradiation, may be rendered more basic in a reversible fashion. A protecting group of a masked base may decompose to reveal a basic nitrogen atom upon exposure to light. Or, a basic nitrogen atom of a molecule obscured by a steric wall may be reversibly swung away in a photochemically-triggered manner. The present invention shows the intrinsic basicity of a nitrogen-containing functional group may be altered by a photochemical event.

Methods of breaking down triazabutadiene molecules may feature subjecting the molecule to light. The light may, for example, include wavelengths of about 400 nm. The present invention is not limited to wavelengths of 400 nm or about 400 nm. For example, in some embodiments, the wavelength is from 350 nm to 400 nm (e.g., 370 nm). In some embodiments, the wavelength is from 360 nm to 410 nm. In some embodiments, the wavelength is from 330 nm to 420 nm. In some embodiments, the wavelength is from 340 nm to 430 nm. In some embodiments, the method comprises subjecting the molecule to a low pH and to light.

As previously discussed, light-promoted reactivity and light-facilitating E/Z isomerization has been observed. In some embodiments, a system such as a UV-LED pen may be used for these reactions, however the present invention is not limited to a UV-LED pen and may utilize any appropriate system. The UV-LED pens may allow for relatively narrow bandwidth irradiation of these compounds (but are not limited to these bandwidths). The color of the bulk material shifts as a result of electronic perturbations to the aryl azide starting material. These experiments may be performed in basic aqueous solutions to maintain the solvation properties of water while also preventing the degradation pathway stemming from protonation. These experiments are not limited to basic aqueous solutions. Without wishing to limit the present invention to any theory or mechanism, it may be considered advantageous that the breakdown of the triazabutadiene molecule is irreversible.

III. Synthesis of Water-Soluble Triazabutadiene Molecules and Experimental Examples

Synthesis of 1-mesityl-1-H-imidazole: To a solution of 2,4,6-trimethylaniline (1.35 g, 10.0 mmol) in methanol (15 mL) [symbol] was added a solution of glyoxal (40%) (1.14 mL, 40% in water, 10. mmol). The mixture was stirred at room temperature until a solid formed. Thereafter, solid ammonium chloride (1.07 g, 20 mmol), formaldehyde (37%) (1.6 mL 37% in water, 60. mmol) and methanol (40 mL) were added, and the mixture was heated to reflux for one hour. After the hour, phosphoric acid (1.4 ml of an 85% solution) was added drop wise and the mixture was refluxed for an additional eight hours. Upon cooling to room temperature ice (30 g) was added and the solution was brought to a pH of 9 with potassium hydroxide (40% in water). The following mixture was extracted repeatedly with diethyl ether. The ether phase was dried over magnesium sulfate and solvent removed in vacuo to form a brown solid which was filtered and washed with hexanes to give the product (0.785 g; 42%). 1 H NMR (500 MHz, CDCI3): δ 7.45 (t, J=1.1 Hz, 1 H), 7.25 (t, J=1.1 Hz, 1 H), 6.99 (dp, J=1.3, 0.7 Hz, 2 H), 6.91 (t, J=1.3 Hz, 1 H), 2.36 (t, J=0.7 Hz, 3 H), 2.01 (t, J=0.6 Hz, 6 H). 13 C NMR (126 MHz, CDCI3) δ 138.80, 137.47, 135.42, 133.40, 129.55, 128.96, 120.02, 21.03, 17.33. (see Liu, J. et al. Synthesis 2003, 17, 2661-2666).

Synthesis of 3-(1-mesityl-1 H-imidazol-3-ium-3-yl) propane-1-sulfonate: To a solution of 1-mesityl-1-H-imidazole (1.00 g, 5.36 mmol) in toluene (30 mL) was added 1,3-propanesultone (1.00 g, 8.18 mmol) and the mixture was heated to reflux overnight. The mixture was allowed to cool to room temperature and the off-white precipitate collected by filtration. The precipitate was further washed with diethyl ether and dried using a vacuum oven to yield a solid (1.40 g; 84%). 1 H NMR (500 MHz, D2O): δ 8.92 (t, J=1.6 Hz, 1 H), 7.75 (t, J=1.8 Hz, 1 H), 7.49 (t, J=1.8 Hz, 1 H), 7.06 (q, J=0.8 Hz, 2 H), 4.44 (t, J=7.1 Hz, 2 H), 2.39 -2.31 (m, 2 H), 2.25 (s, 3 H), 1.96 (s, 6 H). 13 C NMR (126 MHz, D2O) δ 141.42, 136.54, 134.64, 130.74, 124.34, 123.00, 48.18, 47.17, 25.03, 20.17, 16.29.

Synthesis of Potassium 3-(3-mesityl-2-(phenyltriaz-2-en-1-ylidene)-2, 3-dihydro-1 H-imidazol-1-yl) propane-1-sulfonate: To a slurry of 3-(1-mesityl-1H-imidazol-3-ium-3-yl)propane-1-sulfonate (50 mg, 0.16 mmol) in dry THF (6 mL), was added a solution of phenyl azide in THF (0.16 mL, 1 M, 0.16 mmol). To the solution was added KO-t-Bu (24 mg, 0.21 mmol) in one portion and the resulting mixture was stirred under argon for 4 hours. Hexanes (1 mL) was then added and the reaction mixture was filtered. The solvent was removed and the residue taken up in a minimal amount of DCM and on trituration with hexanes, pure product was obtained by filtration as a yellow powder (61 mg, 81%). 1 H NMR (500 MHz, DMSO-d6) δ 7.32 (d, J=2.4 Hz, 1 H), 7.07-7.02 (m, 4 H), 6.99-6.94 (m, 1 H), 6.84 (d, J=2.4 Hz, 1 H), 6.51-6.47 (m, 2 H), 4.09 (t, J=7.1 Hz, 2 H), 2.34 (s, 3 H), 2.12-2.04 (m, 2 H), 1.95 (s, 6 H). 13 C NMR (126 MHz, DMSO-d6) δ 152.19, 151.13, 137.94, 136.15, 134.31, 129.31, 128.60, 125.26, 120.90, 117.61, 117.24, 48.52, 45.05, 25.80, 21.06, 17.95. Using the procedures described herein, the p-methoxy and p-nitro analogs (from the p-MeO aryl azide and p-NO2 aryl azide) were also prepared.

For decomposition experiments, buffers were made to the appropriate pH in a 9:1 mix of H2O:D2O. These solutions were added to the compound being assayed such that the buffer capacity was at least 10 fold the concentration of the compound. Some experiments used 5 mg of the compound in 0.5 mL of buffer. These were immediately inserted into an NMR instrument and scans were taken at even time intervals to calculate the half-life of the compound based on integration.

As another non-limiting example, an azide (e.g., NHS-azide) to N-heterocyclic carbene (NHC) route may be used to synthesize triazabutadiene molecules.

IV. Applications and Methods of Use of Triazabutadienes

The triazabutadiene molecules of the present invention may be utilized for a variety of purposes. For example, in some embodiments, the triazabutadiene molecules of the present invention are utilized for a cleavable linkage (e.g., chemoselectively-cleavable linkage) for use in biological/complex settings where rapid, clean cleavage is of interest. In some embodiments, the triazabutadiene molecules are used for systems including but not limited to drug delivery systems, protein-protein interaction systems, pH environment detection systems, etc. Applications of these triazabutadienes may fall under one (or more) categories of reactivity.

a. Diazonium Coupling Applications and Triazabutadiene Probes

Regarding diazonium coupling, the triazabutadiene molecules may be used for applications involving pH-dependent protein coupling. General examples involve methods for detecting protein-protein proximity or protein-protein interactions (in a sample). In some embodiments, the method comprises providing a first protein, wherein the first protein is conjugated with a triazabutadiene molecule according to the present invention. The first protein may be introduced to a sample. In some embodiments, the triazabutadiene molecule encounters a low pH in the sample; in some embodiments, acid is added to the sample to lower the pH appropriately. As previously discussed, in the low pH environment, the triazabutadiene molecule undergoes the irreversible reaction yielding the diazonium species and the cyclic guanidine species. As previously discussed, the diazonium species is adapted to react with a phenol group; thus if there is a nearby protein with a tyrosine residue, the diazonium species may react with it yielding an azobenzene product (often colored, for example the dye, Sudan Orange G is an azobenzene containing dye) that is visually distinct from the triazabutadiene molecule and the diazonium species. As such, detection of the azo dye may be indicative of proximity or interaction of the first protein and the second protein. Thus, in some embodiments, the method comprises adding a second protein to the sample, wherein a tyrosine of the second protein may react with the diazonium species. In some embodiments, the second protein is already in the sample. In some embodiments, a tyrosine or phenol species conjugated to the second protein. In some embodiments, the method comprises introducing to the sample a first antibody specific for a first protein, wherein the first antibody is conjugated with a triazabutadiene molecule according to the present invention. In some embodiments, the method comprises introducing to the sample a second antibody specific for a second protein. In some embodiments, the second antibody comprises a tyrosine. In some embodiments, the second antibody is conjugated with a phenol species. In some embodiments, the method comprises introducing an acid to the sample to appropriately lower the pH of the sample. As previously discussed, in the low pH environment, the triazabutadiene molecule undergoes the irreversible reaction yielding the diazonium species and the cyclic guanidine species. As previously discussed, the diazonium species is adapted to react with a phenol group; thus if the phenol species is nearby, the diazonium species may react with it yielding an azo dye that is visually distinct from the triazabutadiene molecule and the diazonium species. As such, detection of the azo dye may be indicative of proximity or interaction of the first protein and the second protein.

As a more specific example, the acid-labile reactivity of triazabutadienes may be used to assist in work deducing interaction partners between a virus and endosomally localized host proteins. Upon endosomal acidification a viral-bound diazonium species may be unmasked and this may go on to react with Tyr-containing proteins that are associated with the virus. It is possible that this system could be used to detect or trap an interaction that is relevant at a key point of viral entry, e.g., the fusion of membranes.

Herein are non-limiting examples of synthesis of compounds that may be used in such systems, e.g., for modifying the viral surface. Lysine-reactive probes may be used to modify the surface of proteins. As previously discussed, a triazabutadiene molecule may be attached to a viral protein (e.g., a purified viral protein). Then, a system such as a cell line (e.g., mosquito cell line, human cell line, or even mosquitos themselves) may be infected with the viral protein. The infected system can be treated appropriately. The azo dye (e.g., Sudan Orange) may “label” any proteins that interact with or are nearby the viral protein (in the low pH environment). The present invention is not limited to this example. Lys-NHS conjugation chemistry may work well on the basic side of neutral, which may be beneficial for pH sensitive probes.

As previously discussed, the present invention includes triazabutadienes that function as cross-linkers, e.g., cleavable cross-linkers. In some embodiments, the triazabutadiene cross-linkers allow for linking components via click chemistry, e.g., via copper-catalyzed azide-alkyne cycloadditions. For example, if a clickable handle (e.g., a terminal alkyne handle) is disposed on the triazabutadiene, it can be used to undergo 1,3-dipolar cycloaddition with an azide handle on a different component (e.g., to yield a 1,4-disubstituted triazole).

The use of triazabutadienes and click chemistry allows for the linking of a wide range of compounds for either chemical or biological applications. Note that in general, in order for the azide-alkyne cycloaddition to occur, it must be activated with a Cu(I) source. In some embodiments, the Cu(I) initiator can come from copper-halide reagents or Cu(II) sources that are reduced in situ. Cu(II) salts such as CuSO₄ allow click chemistry to proceed in aqueous conditions with mild reducing agents such as sodium ascorbate. Cu(I) halide salts generally require a base/ligand to coordinate the metal insertion and prevent oxidation. Without wishing to limit the present invention to any theory or mechanism, it is believed that copper click chemistry is versatile as it can be performed in a wide range of conditions. This may allow for tunability when it comes to finding the appropriate conditions for triazabutadiene functionalization.

Note that in some embodiments, the alkyne handle is disposed on the triazabutadiene and said alkyne handle can react with an azide handle on a different component. The present invention is not limited to the alkyne handle being deposed on the triazabutadiene. In some embodiments, the azide handle is disposed on the triazabutadiene and said azide handle can react with an alkyne handle on a different component. In some embodiments, both an alkyne handle and an azide handle are linked to the triazabutadiene.

b. Diazonium Degradation for Cargo or Drug Release

In some embodiments, the triazabutadiene molecules of the present invention may be used in applications involving diazonium degradation to release cargo or drugs. For example, a group of applications takes advantage of the solvolysis of diazonium salts to produce phenolic byproducts. The degradation of diazonium salts to phenols, via aryl cations, is a first-order process that is not pH dependent in the physiological range of pHs. The half-life of this first order process depends on substitution on the aryl ring; the rate for benzenediazonium is ˜4 hours. Indeed, the product of this degradation and subsequent azo-dye formation was observed if resorcinol is not put into the buffered NMR experiments.

In some embodiments, the acid-dependent instability of the triazabutadiene molecule may allow for a drug or cargo molecule to be deposited at a desired location and time (e.g., the reaction can be controlled and initiated at a desired time and location). As such, the present invention also features methods of delivering a drug (or a cargo compound) to a subject. In some embodiments, the method comprises providing a triazabutadiene molecule according to the present invention, conjugating a drug (or cargo compound) to the triazabutadiene molecule; and administering the conjugate (the drug/cargo-triazabutadiene conjugate) to the subject. In some embodiments, the method comprises providing a triazabutadiene molecule according to the present invention wherein the triazabutadiene molecule comprises the drug (or cargo compound); and administering the triazabutadiene molecule to the subject. In some embodiments, the diazonium species of the triazabutadiene molecule is part of the drug (or cargo compound). In some embodiments, the drug (or cargo compound) is formed when the diazonium species reacts to a phenol species. In some embodiments, the drug is an anti-cancer drug. The drug (or cargo compound) is not limited to an anti-cancer drug. Any appropriate drug for any appropriate condition may be considered. Likewise, the triazabutadiene molecules may be incorporated into drug/cargo-delivery systems for conditions including but not limited to cancer or other conditions associated with low pH states (e.g., gastrointestinal conditions, sepsis, ketoacidosis, etc.). Non-limiting examples of drugs (e.g., drugs that have a phenolic functional group, which may be masked as prodrugs) include: Abarelix, Alvimopan, Amoxicillin, Acetaminophen, Arformoterol, Cefadroxil, Cefpiramide, Cefprozil, Clomocycline, Daunorubicin, Dezocine, Epinephrine, Cetrolrelix, Etoposide, Crofelemer, Ezetimibe, Idarubicin, Ivacaftor, Hexachlorophene, Labetalol, Lanreotide, Levodopa, Caspofungin, Butorphanol, Buprenorphine, Dextrothyroxine, Doxorubicin, Dopamine, Dobutamine, Demeclocycline, Diflunisal, Dienestrol, Diethylstilbestrol, Doxycycline, Entacapone, Arbutamine, Apomorphine, Balsalazide, Capsaicin, Epirubicin, Esterified Estrogens, Estradiol Valerate, Estrone, Estradiol, Ethinyl Estradiol, Fulvestrant, Goserelin, Fluorescein, Indacaterol, Levosalbutamol, Levothyroxine, Liothyronine, Lymecycline, Mitoxantrone, Monobenzone, Morphine, Masoprocol, Mycophenolic Acid, Phenylephrine, Phentolamine, Oxytetracycline, Rifaximin, Rifapentine, Oxymetazoline, Raloxifene, Tolcapone, Terbutaline, Tetracycline, Mesalamine, Metaraminol, Methyldopa, Minocycline, Nabilone, Nalbuphine, Nelfinavir, Propofol, Rotigotine, Ritodrine, Salbutamol, Sulfasalazine, Salmeterol, Tapentadol, Tigecycline, Tolterodine, Teniposide, Telavancin, Topotecan, Triptorelin, Tubacurarine, Valrubicin, Vancomycin, etc.

In some embodiments, drug delivery systems featuring triazabutadiene molecules may be enhanced with other reactions, e.g., enzymatic reactions. Such additional reactions may help provide appropriate specificity of the drug delivery system or appropriate timing to the drug delivery system.

The present invention also features a method for administering a drug comprising a phenolic function group to a subject in need of such a drug administration. In some embodiments, the method comprises converting a drug comprising a phenolic-functional group to a prodrug, wherein said prodrug comprises an acid labile triazylidene moiety; and administering said prodrug to a subject in need of such a drug administration. In some embodiments, the triazylidene compound may also comprise a water solubility conferring moiety and/or Y¹ functional group.

The present invention also features a method of converting a drug comprising a phenolic-function group to an acid labile prodrug. In some embodiments, the phenolic-functional group is converted to an azide group. The azide functional group may then be reacted with a carbene to produce an acid labile prodrug comprising a triazylidene moiety.

In some embodiments, a triazabutadiene molecule is conjugated to another molecule (a conjugate molecule), e.g., a protein (e.g., an amino acid such as but not limited to lysine), a lipid, or other appropriate molecule. In some embodiments, the diazonium species part of the triazabutadiene molecule is conjugated to the conjugate molecule. In some embodiments, the cyclic guanidine species part of the triazabutadiene molecule is conjugated to the conjugate molecule. In some embodiments, the triazabutadiene molecule is attached to the conjugate molecule via a linker. Linkers are well known to one of ordinary skill in the art and may include (but are not limited to) polyether linkers such as polyethylene glycol linkers. In some embodiments, the conjugate molecule to which the triazabutadiene molecule is conjugated comprises an antibody or a fragment thereof. In some embodiments, the conjugate molecule to which the triazabutadiene molecule is conjugated comprises a viral protein.

In some embodiments, the triazabutadiene molecules of the present invention are used for pull-down studies wherein a biomolecule or protein of interest is attached to one side and the other side is appended to something such as but not limited to a small molecule (e.g., hapten such as biotin) or compound. Using biotin as an example, the biomolecule or protein of interest can be pulled down using an avidin bead (which binds strongly to the biotin) and thoroughly washed. This may be useful for protein enrichment. The biomolecule or protein of interest may then be cleaved from the avidin bead by means of reductive cleavage of the triazabutadiene that holds them together. The present invention is not limited to these components, for example this application could also feature the use of a probe (e.g., fluorescent or otherwise) attached to an antibody used to interrogate a complex sample.

In some embodiments, reductive cleavage of triazabutadiene molecules may also be used to cleave unreacted triazabutadienes that did not undergo diazonium formation/reaction chemistry that is associated with a drop in pH (or other mechanism) as described above (a sort of quench for the pH chemistry).

As previously discussed, the diazonium species can react with a phenol species such as resorcinol or other appropriate phenol species. In some embodiments, a phenol species or resorcinol species is conjugated to a protein, e.g., a protein different from the protein to which the triazabutadiene molecule is conjugated, a protein that is the same protein to which the triazabutadiene molecule is conjugated, etc. In some embodiments, the resorcinol species or phenol species that the diazonium species reacts with is the phenol functional group of a tyrosine residue.

c. Intracellular Delivery of Diazonium Ions

The present invention describes protected (or masked) triazabutadienes as probes for intracellular experiments and for selective intracellular delivery of diazonium ions (or benzene diazonium ions (BDIs).

Referring to FIG. 3, at physiological pH and upon protonation at the N3 position, triazabutadienes are protonated to release a diazonium ion (e.g., benzene diazonium ion, BDI) and a guanidinium side product.

It was found that formylating the N1 position of triazabutadienes protected the release of the BDI physiological pH and acidic conditions. Upon exposure to a high pH environment, the triazabutadiene is deprotected and once again able to release a BDI (see FIG. 3). While the base-labile protection approach has utility in select settings, the general concept of protecting the N1 position provides a strategy by which a desired selectivity and deliverability can be obtained in a range of settings (see FIG. 3). The present invention also provides methods for utilizing the triazabutadiene protection strategy to deliver a BDI to determine its in cellulo effects. Taking advantage of the high intracellular reducing potential, a reduction-sensitive group was envisioned to offer spatial control of the probe with uncaging of the triazabutadiene moiety initiated only in a reducing intracellular environment.

Referring to FIG. 4A, tert-butyl/methyl triazabutadiene (molecule 2, or “2”) was chosen for one study. The half-life of the molecule was shown to be <2 min at pH 8, thus the BDI release is faster than a phosphorylation assay and the timing of the release of the BDI was dependent upon the deprotection event that would liberate (2) in solution. Various concentrations of (2) were incubated with HEK293T cells for 3 h, the cells were lysed, and global tyrosine phosphorylation levels were measured using a fluorescent 2° p-Tyr antibody. It was surprising that global tyrosine phosphorylation levels had increased in a 2-dependent manner (FIG. 4B). Repeated experiments showed a marked increase in global tyrosine phosphorylation at 500 μM of (2). A time-course experiment incubating HEK293T cells with 500 μM of (2) was conducted, and the increase in the global tyrosine phosphorylation signal was observed as early as 2 h (FIG. 4C).

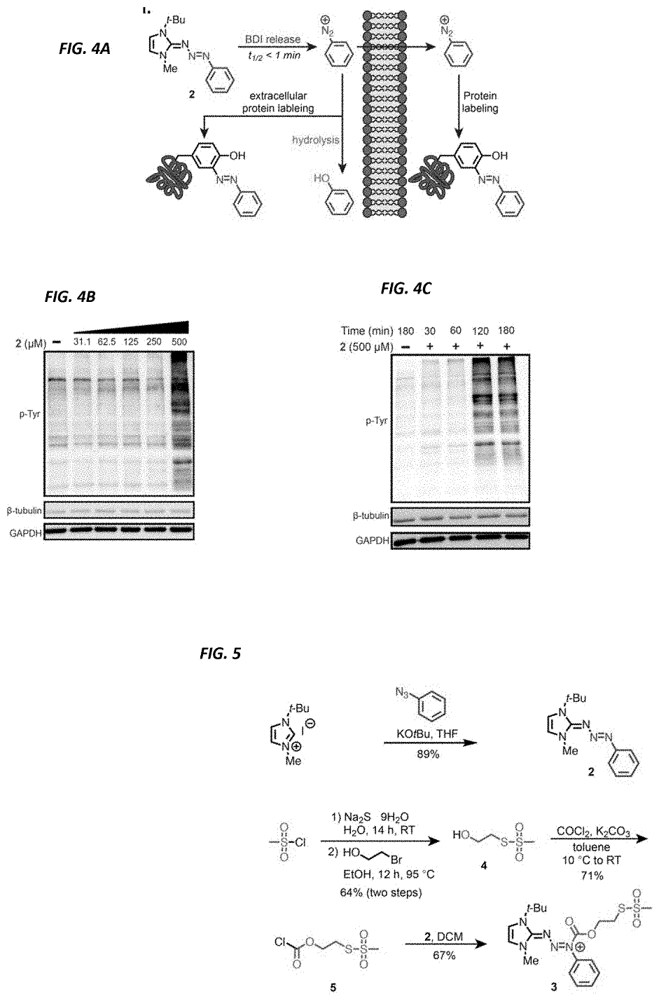

Protected triazabutadienes may be synthesized with a nucleophilic triazabutadiene and a corresponding chloroformate electrophile. The unprotected tert-butyl/methyl triazabutadiene, (2), was synthesized in two steps from tert-butyl imidazole and phenyl azide (FIG. 5). For the chloroformate electrophile, alcohol (4) was synthesized in two steps from previously established methods and (4) was treated with phosgene to yield chloroformate (5). The reaction between triazabutadiene (2) and chloroformate (5) provided the protected triazabutadiene (3) in reasonable yield (FIG. 5). Unlike (2), protected 3 is stable for days in neutral water. In growth media containing resorcinol as a BDI trap, (2) readily provided the corresponding azobenzene product, and (3) remained intact until dithiothreitol was added to trigger deprotection and BDI release.

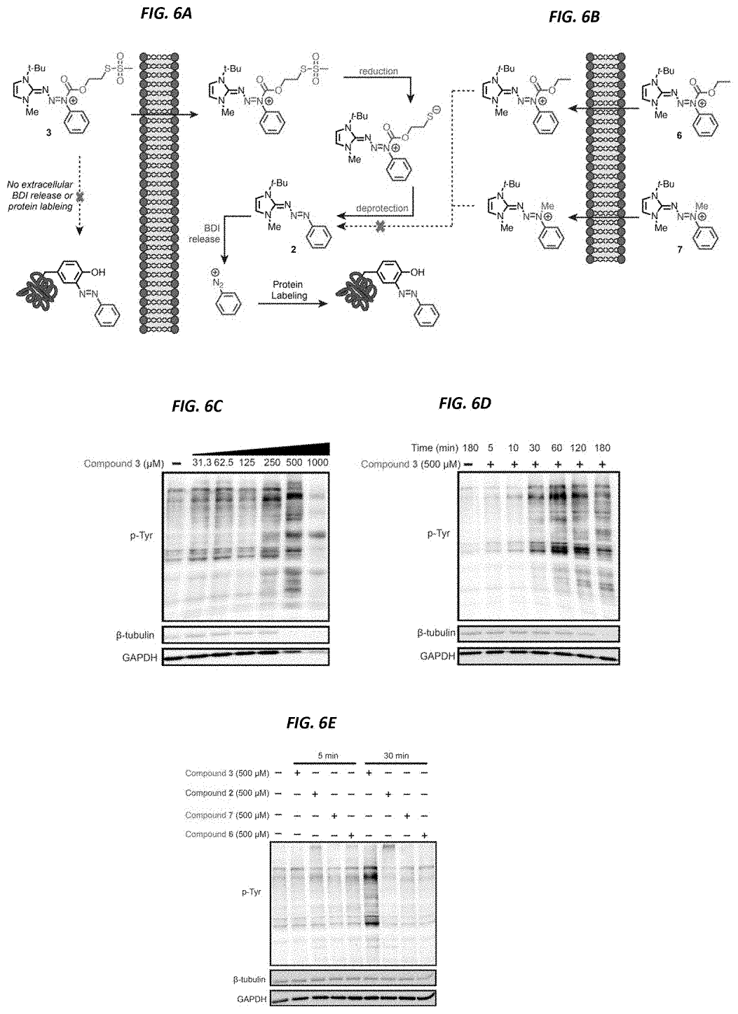

In addition to (3), two control compounds were also synthesized (FIG. 6A, FIG. 6B). Compound 6 is an ethyl carbamate protected triazabutadiene, which can deprotect and release a BDI only after exposure to a high pH environment. This compound helps to rule out an extracellular release mechanism of (3). The second control compound, (7), is a triazabutadiene that was alkylated at the N1 position.

Concentration and time-dependent experiments were performed with (3). It was observed that (3) considerably increased global tyrosine phosphorylation at 500 μM; however, this increase was also observed to be accompanied by a loss of the β-tubulin and GAPDH housekeeping proteins (FIG. 6C). Additionally, upon using (3) in time-course experiments, the increase in global tyrosine phosphorylation was observed as early as 30 min, consistent with the previously observed rates of similar self immolative compounds (FIG. 6C, FIG. 6D). Upon directly comparing (2), (3), (6), and (7), there was no effect on global phosphorylation observed for either (6) or (7) at 30 min or up to 180 min (FIG. 6E) further bolstering the model of an intracellular BDI being potentially responsible for the observed increase in global tyrosine phosphorylation.

An in vitro DiFMUP phosphatase inhibition assay was conducted measuring phosphatase activity as a function of fluorescence. After adding (2) to a solution, it generates (1) rapidly. As such, for these assays, (2) was used to determine the effects the BDI had on phosphatases. It was observed that treatment of PTP1B with (2) inhibits PTP1B with an IC50 of 2.5 μM.

The inhibition assay was repeated with PP1, an unrelated serine/threonine phosphatase, and it was observed that PPI was also inhibited with an IC50 of 3.8 μM.

Samples were subjected to proteomics analysis to identify the specific residues that had been modified by (1). Experiments were run with 10 μM 2 and 20 nM PTP1B, following the same conditions as the in vitro inhibition assays with PTP1B. The samples were subjected to trypsin digestion and subsequent proteomic analysis with mass spectrometry. A spectrum counting analysis was first conducted with the Scaffold program to evaluate for the presence of modifications. It was observed that many surface exposed, or “easily accessible”, tyrosine and histidine residues on PTP1B were not modified by the BDI. Only six tyrosine and histidine residues were observed to have undergone modification: H54, H60, Y66, H94, Y176, and H214. While the azobenzene was seen to be intact on some residues, others were presented as a cleaved modification. The extracted ion abundance of these modifications was then quantified using the program Progenesis QI for Proteomics. It was found that compared to a control sample H54, H60, and Y66 all had a statistically significant increase in azobenzene modification upon the addition of the BDI (p<0.01).

The present invention provides a delivery mechanism for BDIs, which may function as bioconjugation agents for profiling surface tyrosine and histidine residues. The protected triazabutadiene technology described herein has shown to be beneficial in delivering the BDI intracellularly. This represents the first known report of triazabutadienes being utilized for in cellulo experiments, and the first known report of an aryl diazonium ion being targeted intracellularly.

The present invention provides a platform for a wide range of studies. The ability to selectively bring reactive electrophiles to specific biochemical environments enables a range of experiments, wherein the accessibility and reactivity of various residues can be interrogated in intact biological systems. This claim is strengthened by the selectivity observed in the mass spectrometry experiments, providing support for a model where the resulting BDI from (2), or variants thereof, could be used as a small molecule covalent probe for activity-based protein profiling (ABPP). Functional side chain profiling has been done with cysteine, lysine, and tyrosine; additional methods are being explored in order to profile functional histidine residues, e.g., alkyne-containing diazonium ions used for proteomic profiling of cell lysate, the global cellular effects of treatment with the BDI using pull-down probes and whole-cell proteomics, etc. The present invention also includes methods for exploring cellular targets that the BDI modifies.

FIG. 7 shows a non-limiting example of the structure of a protected triazabutadiene molecule. In some embodiments, X and/or Y are chosen to modulate the rate of release at a given pH. In some embodiments, X and/or Y can be chosen to aid in delivery of the molecule (e.g., triphenylphosphonium can direct the molecule to the mitochondria, folate can be added to induce cellular uptake via the folate receptor, etc). Z enables linkage to other small molecules or proteins. In some embodiments, Z is a Cu-click reagent, a Cufree (SPAAC) reagent, another bioorthogonal functionality, etc., e.g., a drug or drug-like moiety to promote protein binding. In some embodiments, Z is an NHS-ester to enable conjugation to functionalized amines. Q is a location-specific immolative linker. In some embodiments, Q is made to provide —(CH₂)₂S—, variants that employ Thorpe-Ingold effect for rate enhancement, a molecule that promotes Grob fragmentation (e.g., TMS), etc.. In some embodiments, Q can also provide a quinone-methide to eliminate. In some embodiments, R is a heteroatom bound to a substrate that is cleaved (e.g., cleaved enzymatically).

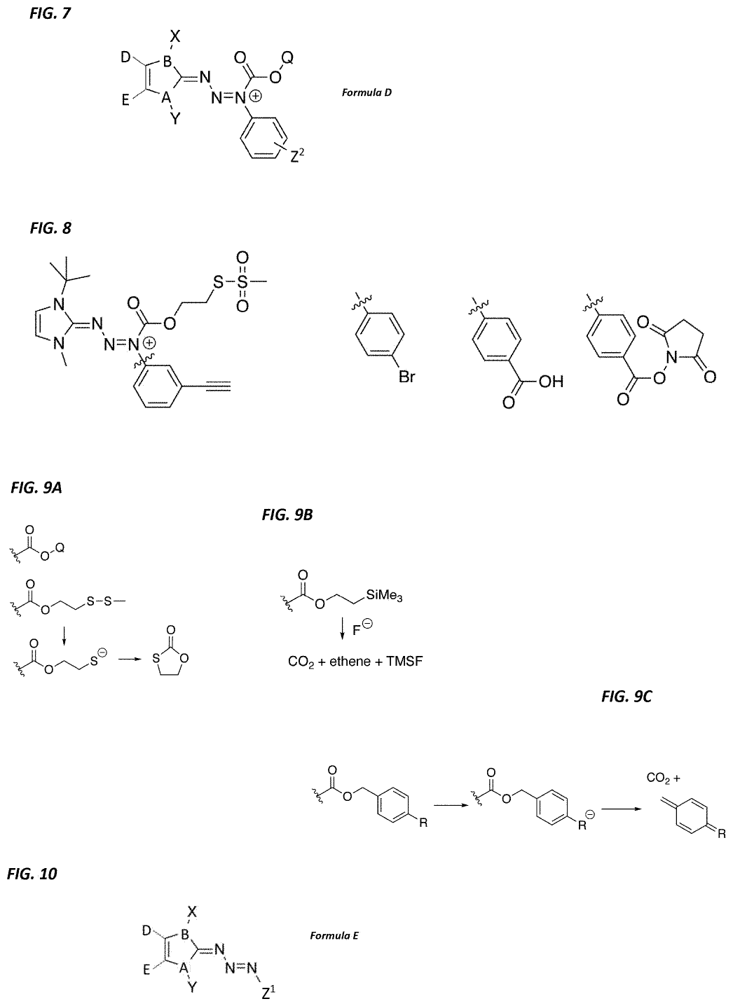

FIG. 8 shows non-limiting examples of protected triazabutaidene molecules that enable further conjugation to other molecules, e.g., biomolecules. FIGS. 9A, FIG. 9B, and FIG. 9C show non-limiting examples of Q and a range of mechanisms by which triazabutadiene protecting groups can be removed.

### EXAMPLE

The following is a non-limiting example of the present invention. It is to be understood that said example is not intended to limit the present invention in any way. Equivalents or substitutes are within the scope of the present invention.

Synthesis of (2-((Chlorocarbonyl)oxy)ethyl) Methanesulfonothioate (5). To a flame-dried flask was added 100 mg of crushed 4 Å molecular sieves. Then, a solution of K2CO3 (1.3 mmol) in toluene (4 mL) was added under argon. The solution was cooled to −10° C. and(2-hydroxyethyl) methanesulfonothioate (1.1 mmol) was added to the reaction vessel slowly. Then, a 20% solution of phosgene in toluene (1 mmol) was added dropwise to the solution for over 10 min. The reaction mixture was allowed to stir for 30 min at −10° C. and then at room temperature for 4 h under argon. After 4 h, argon was bubbled through the reaction mixture for 5 min in a closed hood with an outlet in the septa to remove excess phosgene. The reaction was filtered over a pad of MgSO4 and washed with ether. The resulting filtrate was evaporated to dryness to yield 5, which was taken forward and used without further purification.

(E)-3-(tert-Butyl)-1-methyl-2-(3-((2((methylsulfonyl)thio)ethoxy)carbonyl)-3-phenyl triaz1-en-1-yl)-1 H-imidazol-3-ium chloride (3) synthesis. A flame-dried and vacuum-evacuated flask under argon was charged with 4 Å molecular sieves and dichloromethane. To this solution was added 5 (0.8 mmol). This solution was allowed to stir under argon for 5 min. To this was added 2 (0.07 mmol) in one portion at room temperature. The reaction mixture was left to stir under argon for 12 h, after which time it was filtered, and the filtrate was concentrated down to yield a yellow solid. Purification of 3 involved a silica column with 10% MeOH/CH2Cl2. Following that, the resulting product was dissolved in CH2Cl2 and washed three times with aqueous 0.1% TFA solution. The CH2Cl2 layer was evaporated to dryness to provide 3 as a yellow solid (0.021 g, 67% yield).

In Cellulo Global Tyrosine Phosphorylation Assays. HEK 293T cells were maintained in complete media (90% DMEM, 10% FBS, 100 U/mL penicillin, 100 μg/mL streptomycin, and 2.5 μg/mL amphotericin B). Cells were plated 24 h prior to treatment. 24 h post cell plating, cells were treated with compounds or DMSO for a specified amount of time. Cells were incubated for the indicated times with appropriate compounds and then lysed with MPER supplemented with proteasome and phosphatase inhibitors for 20 min at 4° C. while gently agitating. Cell lysates were then centrifuged at 14,000 rcf for 10 min at 4° C. Protein concentration of the supernatant was quantified using the BCA reagent, and 30 μg of total protein per well was loaded on SDS PAGE gels. Proteins were transferred onto PVDF membranes and blocked using 5% BSA in TBST for 1 h at room temperature. Membranes were probed with primary antibodies of interest overnight at 4° C. while gently agitating. Membranes were then washed and probed with secondary antibodies for 1 h at room temperature. Membranes were then imaged using a BioRad ChemiDoc MP imaging system.

In Vitro Phosphatase Assays. All DiFMUP fluorogenic based phosphatase assays were carried out at least in duplicate. All assays were carried out in a final volume of 100 μL. Enzymes (PP1 or PTP1B) were incubated in reaction buffer [50 mM Tris solution pH 7, 200 μM MnCl2, 2 mM DTT, 0.05% (v/v) Tween-20, and 125 μg/mL BSA] with DMSO only or with compounds at specific times before the addition of 100 μM DiFMUP. DiFMUP fluorescence over time was measured using a BMG LABTECH CLARIOstar Plus microplate reader. Samples were excited at 358 nm and emission scans recorded from 420 to 470 nm with a maximum emission at 448 nm.

Mass Spectrometry Analysis of BDI Conjugation. Experiments were carried out in duplicate in a final volume of 100 μL. 1 μg of PTP1B was incubated in reaction buffer [50 mM Tris solution pH 7, 200 μM MnCl2, 2 mM DTT, 0.05% (v/v) Tween-20, and 125 μg/mL BSA] with controls of DMSO only or replicates of 2. Samples were trypsin-digested, purified as previously described, and subsequently analyzed by LC-MS/MS as previously described.

The disclosures of the following documents are incorporated in their entirety by reference herein: U.S. Pat. No. 8,617,827; U.S. Pat. Application No. 2009/0048222; U.S. Pat. No. 3,591,575. U.S. Pat. No. 3,607,542; U.S. Pat. No. 4,107,353; WO Pat. No. 2008090554; U.S. Pat. No. 4,218,279; U.S. Pat. App. No. 2009/0286308; U.S. Pat. No. 4,356,050; U.S. Pat. No. 8,603,451; U.S. Pat. No. 5,856,373; U.S. Pat. No. 4,602,073; U.S. Pat. No. 3,959,210. The disclosures of the following publications are incorporated in their entirety by reference herein: Kimani and Jewett, 2015, Angewandte Chemie International Edition (DOI: 10.1002/anie.201411277 —Online ahead of print). Zhong et al., 2014, Nature Nanotechnology 9, 858-866; Stewart et al., 2011, J Polym Sci B Polym Phys 49(11):757-771; Poulsen et al., 2014, Biofouling 30(4):513-23; Stewart, 2011, Appl Microbiol Biotechnol 89(1):27-33; Stewart et al., 2011, Adv Colloid Interface Sci 167(1-2):85-93; Hennebert et al., 2015, Interface Focus 5(1):2014.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A composition for selective intracellular delivery of a diazonium species.

2. The composition of claim 1, wherein the diazonium species labels a tyrosine or histidine residue of a protein in cellulo.

3. The composition of claim 1, wherein the composition is for use in cellulo, and the composition is configured to selectively release a diazonium species upon exposure to a high pH.

4. The composition of claim 3, wherein the high pH is a pH of 9 or higher.

5. The composition of claim 3, wherein the composition can be taken up intracellularly.

6. A composition is according to Formula D: 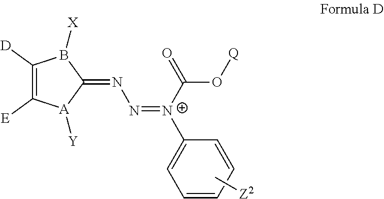
wherein
Z² is a NHS ester, a Cu click reagent, a Cu free click reagent, a bioorthogonal handle, or a drug; and
Q is —(CH₂)₂S—, an enzymatically cleavable moiety, a self-immolative linker, a quinone methide forming cascade reaction, or a Grob-fragmentation related cleavable linker.

7. The composition of claim 6, wherein:
A is N, S, or O;
B is N, S, or O;
D is H, —CH=CH—CH=E—, halides, cyano, sulfonates, alkyl chain, or trifluoromethyl;
E is H, —CH=CH—CH=D—, halides, cyano, sulfonates, alkyl chain, or trifluoromethyl;
X is —R1-K1, wherein —R1=alkanes and K=sulfonate, phosphate, or a quaternary ammonium cation, or an alkyl, aryl or propargylic containing moiety that can facilitate coupling to other azides via [3+2]cycloaddition chemistry, or non-existent if B is S; and
Y is a tri-substituted aryl group or an alkyl substituent or non-existent if A is S.

8. The composition of claim 7, wherein one, or a combination of, or all of X, Y, and Q comprise a biological directing group.

9. The composition of claim 8, wherein the biological directing group is a triphenylphosphonium for directing the composition to the mitochondria or a folate for inducing cellular uptake via folate receptor.

10. The composition of claim 7, wherein the tri-substituted aryl group of Y comprises mesityl, a NHS-ester moiety; an oligonucleotide; a peptide; a fluorescence quencher; a pro-fluorophore; an alkyne; a triazene; an aldehyde; an amine; an aminooxy; a halogen; or a combination thereof.

11. A composition is according to Formula E: 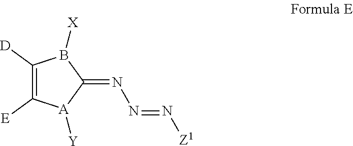
wherein
A is N, S, or O;
B is N, S, or O;
D is H, —CH=CH—CH=E—, halides, cyano, sulfonates, alkyl chain, or trifluoromethyl;
E is H, —CH=CH—CH=D—, halides, cyano, sulfonates, alkyl chain, or trifluoromethyl;
X is —R1-K1, wherein —R1=alkanes and K=sulfonate, phosphate, or a quaternary ammonium cation, or an alkyl, aryl or propargylic containing moiety that can facilitate coupling to other azides via [3+2]cycloaddition chemistry, or non-existent if B is S;
Y is a tri-substituted aryl group or alkyl substituents or non-existent if A is S; and
Z¹ is a polymerization residue, a phenyl group, a substituted phenyl group, or —COO—Q.

12. The composition of claim 11, wherein the tri-substituted aryl group of Y comprises mesityl, a NHS-ester moiety; an oligonucleotide; a peptide; a fluorescence quencher; a pro-fluorophore; an alkyne; a triazene; an aldehyde; an amine; an aminooxy; a halogen; or a combination thereof.

13. The composition of claim 11, wherein Z¹ comprises both a phenyl group or phenyl-Z² and —COO—Q, each being bound to N1 nitrogen.

14. The composition of claim 13, wherein Z² is a NHS ester, a Cu click reagent, a Cu free click reagent, a bioorthogonal handle, or a drug.

15. The composition of claim 11, wherein Q is —(CH₂)₂S—, an enzymatically cleavable moiety, a self-immolative linker, a quinone methide forming cascade reaction, or a Grob-fragmentation related cleavable linker.

16. The composition of claim 11, wherein Z¹ is a pair of side groups that causes N1 to have a positive charge.
